(19) United States
(12) Patent Application Publication
(10) Pub. No.: US 2021/0128062 A1
(43) Pub. Date: May 6, 2021

(54) WEARABLE DEVICE WITH MODELS FOR LIFESTYLE MANAGEMENT

(71) Applicant: T-Mobile USA, Inc., Bellevue, WA (US)

(72) Inventors: Yasmin Karimli, Kirkland, WA (US); Jonathan England, Maple Valley, WA (US); Kristiana M. Lockman, Seattle, WA (US); Kelsey Burns, Seattle, WA (US); Tara Anderson, Seattle, WA (US)

(73) Assignee: T-Mobile USA, Inc., Bellevue, WA (US)

(21) Appl. No.: 16/675,019

(22) Filed: Nov. 5, 2019

(51) Int. Cl. A61B 5/00; G16H 50/20; G16H 50/30; A61B 5/0205

(52) U.S. Cl. CPC A61B 5/4884; G16H 50/20; A61B 5/0205; A61B 5/0022; G16H 50/30

(57) ABSTRACT

User devices can include a lifestyle analyzer component to capture user data to monitor health conditions of the user to provide a personalized lifestyle manager. In some instances, health data from a wearable device can be cross correlated to other application data such as calendar items, communications, location data, social media data, and financial data. Based on negative health metrics (e.g., stress level increase, sleep deprivation, etc.), the lifestyle manager may provide directives to automatically manage communications, schedules, and/or tasks. The user device can capture user data and transmit the data to a serving device to aggregate the data. The serving device can use the aggregated data to perform lifestyle analysis to generate directives for lifestyle management and rules to implement the directives. If the user accepts a directive, the lifestyle manager may implement the rules on a user device.

### BACKGROUND

Many wearable devices for monitoring the activities of a user are known. Such devices generally include a sensor that collects data which is stored on the device and later uploaded to a computing device, transmitted via a link, such as a Bluetooth® connection, to a cellular telephone, and/or transmitted via the device's cellular network connection to a server. These wearable devices typically include a limited, defined set of abilities, such as a pedometer, and are generally designed to serve specific purposes. As a result, the devices are usually at most minimally customizable by the user.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 illustrates an example environment including user devices that include a lifestyle analyzer component to facilitate collection of user data for a personalized lifestyle manager component, as described herein.

FIG. 2 illustrates an example user device configured to implement the personalized lifestyle manager component, in accordance with embodiments of the disclosure.

FIG. 3 illustrates an example serving device configured to receive user data captured by a user device that includes a lifestyle analyzer component, as discussed herein.

FIG. 4 illustrates an example visualization of a personalized lifestyle manager component based on user data captured using a lifestyle analyzer component, as discussed herein.

FIG. 5 illustrates an example process for receiving user data from a wearable device and a user equipment, determining a stress level in response to an electronic communication, and generating a communication filter rule in response, as described herein.

FIG. 6 illustrates an example process for transmitting user data to a serving device and receiving directives to implement the personalized lifestyle manager component, as described herein.

### DETAILED DESCRIPTION

Systems, devices, and methods are directed to user devices including a lifestyle analyzer component to capture user data including health data and application data, and a serving device for analyzing the user data to generate a lifestyle manager component. To provide a customizable experience for a wearable device, additional user data apart from sensor data gathered by the wearable device may be needed. This additional user data may be correlated with sensor data to provide context to the sensor data as it is being tracked. However, even with context added to sensor data, customizing the user experience requires a deeper understanding of the additional user data and using this deeper understanding to intelligently recommend lifestyle management for individual user.

In some examples, health data captured by sensors on a wearable device can be cross correlated or otherwise mapped to other user data from applications including calendar items, communications (e.g., phone calls, emails, etc.), location data, social media data, image data, streaming media data, and financial data. Based on determined health metrics (e.g., heart rate, stress level, hours slept, steps taken, active calories burned, etc.), the lifestyle manager may provide directives to automatically manage communications, schedules, and/or tasks. In various examples, the user may select lifestyle management goals (e.g., fitness increase, productivity increase, financial health, etc.) to better tailor directives for the user. A service provider associated with the serving device may provide the lifestyle analyzer component to continuously capture user data from wearables, user accounts, and user devices to facilitate identification of areas of strong association and/or correlation between health data and general user data to recommend lifestyle changes. In some instances, the user device can capture user data which can be provided to a serving device to determine aggregated user data. The serving device can use the aggregated user data to generate predictive modeling to make directives, recommendations, or other messages for a user in response to the individual user data.

In some instances, the lifestyle analysis may include determining health metrics based on health data received from a wearable device associated with a user and/or a user profile. The health metrics may include stress level, sleep, steps taken, and calories burned. In examples, the stress level may be based in part on changes in heart rate. In some examples, the stress level may be further based on data received from other user device (e.g., user's voice, user's words, user's facial expression, etc.). In additional examples, the stress level based on heart rate may be ignored if the user is exercising. The stress level may be compared to a threshold (e.g., stress level>50) or may be based on a spike (e.g., stress level increases by more than 20 units within a minute (or any other amount and/or period of time)) to determine whether the user is experiencing stress. In various examples, the system may provide a user interface to allow the user to manually self-report on health metrics, including but not limited to, general well-being, mood, stress level, energy level, and the like. The self-reported metrics may be any level value (e.g., on a scale of 1-5). If the user is experiencing stress, the system may analyze other user data received during this time and determine if there is a correlation between the stress level and other user data. For example, the system may determine that the user exhibits a stress level spike every day during their commute at particular intersection. In some instances, the system may provide an alternate route suggestion to bypass that particular intersection even if the alternate route may not necessary be faster. In another example, the system may determine from health metrics that the user is not getting enough sleep and is showing signs of stress before bedtime. The system may download meditation application and/or present breathing techniques for the user before bedtime. The example remediation techniques are examples and additional techniques are discussed herein.

In some instances, the lifestyle analyzer component can be implemented as an application or operating system component executing on the user device configured to interact with other applications of the user device. The lifestyle analyzer component can instruct the operating system component to control software and/or hardware associated with the user device to monitor health data, determine health metrics, application data, and other data at the user device and provide user data (e.g., user location, heart rate, communication status, attending a calendar item, etc.) indicative of the user lifestyle to a serving device for subsequent analysis.

By way of example and without limitation, the techniques discussed herein can be implemented on a user device configured to capture and report user data to a serving device associated with a service provider in response to user subscribing to the service provider's network and/or lifestyle management service. In various examples, the user device may continuously transmit data to the service provider's network. In some examples, a user device not connected to the service provider's network or a local Wi-Fi may transmit data to another user device that is connected to the service provider's network. For instance, if a user's wearable device is not connected to the service provider's network but is within Bluetooth range of the user's phone which is connected to the network, the wearable device may transmit the data to the phone, and the phone may upload the data to the service provider's serving device. The serving device collects the user data and analyzes aggregated data to provide a personalize lifestyle management service to a user.

The systems, devices, and techniques described herein can improve a functioning of a wearable device by capturing user data from multiple user devices to identify areas of strong correlation between health data and general user data to provide a personalized wearable device. This correlated data may be used to generate rules and filters for communications, or provide recommendations based on predictive modeling. As a result, a user is subjected to less stress over communications they may not wish to engage is while the network is freed from handling unwanted communication traffic. For example, the systems, devices, and techniques can determine a stress level spike for a user when receiving communications during a specific time to identify instances where communications could be screened or filtered. Additionally, the systems, devices, and techniques discussed herein provide a distributed framework for user data gathering and analysis, which may dynamically change the data transmission based on user devices and network availability. Moreover, by constantly monitoring the user data, the serving device may automatically and remotely send updated rules for data collecting and for lifestyle management, if needed. Additionally, the updated rules may effectively filter and/or block unwanted communications or internet use which reduces network traffic and battery drain on the user devices. These and other improvements to the functioning of a computer and network are discussed herein.

The systems, devices, and techniques described herein can be implemented in a number of ways. Example implementations are provided below with reference to the following figures.

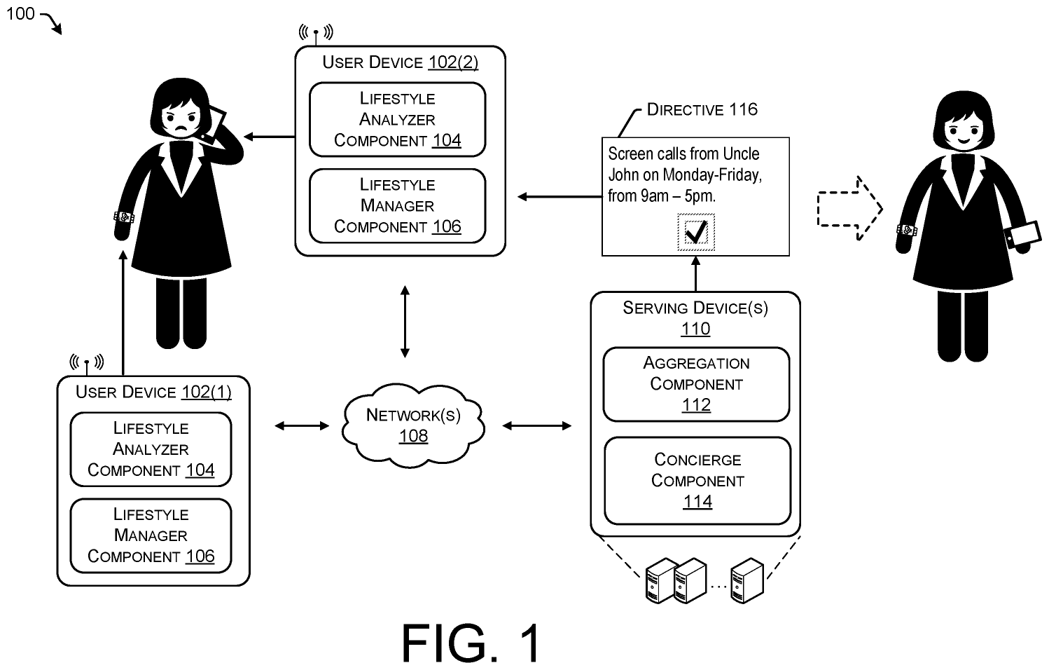

FIG. 1 illustrates an example environment 100 including user devices that include a lifestyle analyzer component to facilitate collection of user data for a personalized lifestyle manager component, as described herein.

In some instances, a user device 102(1) and a user device 102(2) (collectively referred to as user devices 102) can include a lifestyle analyzer component 104 and lifestyle manager component 106. In general, the user device 102(1) and the user device 102(2) can receive user data from applications, sensors, or communications. The applications may include any category of applications including calendar, email, phone book, messenger, maps, social networking, games, media player, finance, fitness, lifestyle, shopping, and the like. The sensors may include heart monitor, blood oxygen monitor, blood pressure monitor, scale, barometer, temperature sensor, pedometer, accelerometer, GPS, camera, microphone, and the like. The communications may be included with applications and may be functions native to phones including voice communications, video communications, texts, and the like.

In some instances, the user device(s) 102 can analyze the user data, as discussed herein, independent of conducting any communications via the network(s) 108 with the serving device(s) 110. As can be understood, the example environment 100 is not limited to the user device 102(1) and user device 102(2) for data gathering, and any number of device(s) can be utilized, as discussed herein. For instance, the functionality of both user device 102(1) and user device 102(2) may be implemented as a single user device 102 such as a smart watch capable of tracking health metrics, placing calls, and storing various applications (e.g., email, calendar, phone book, banking applications, etc.). Additionally, as can be understood, references of a “user” may be the user and/or user profile associated with the user. For instance, a device may be associated with a user profile and health metrics collected by the device may be associated with the user profile and also the user by association.

The lifestyle analyzer component 104 can include functionality to receive and process sensor data from one or more sensors, as discussed herein. The sensor data may be received from one or more sensor that provide information on health metrics associated with a user and/or user profile. In some instances, the lifestyle analyzer component 104 can use one or more sensors to measure health data for various factors (e.g., general fitness, heart rate, stress level, sleep health, steps, calories burned, etc.) that affect a health and lifestyle of a user and/or user profile.

In some examples, the lifestyle analyzer component 104 may determine baseline health metrics for the user and/or user profile. The lifestyle analyzer component 104 may determine a resting heart rate and/or a baseline stress level. The lifestyle analyzer component 104 may determine expected sleep schedule from user input or from user's actual sleep data. The lifestyle analyzer component 104 may store the baseline health metrics for comparison to incoming sensor data to determine if the user is currently (at or near real-time) exhibiting negative health metrics (e.g., stress level spike, high stress level, irregular heart rate, etc.). The lifestyle analyzer component 104 may determine a stress level spike based on an increase of stress level in a short time period (e.g., increase in stress level by more than 20 in a minute) and may determine a high stress level based on a stress level being above a threshold stress level or being over baseline stress level by some threshold. In some examples, the lifestyle analyzer component 104 may determine additional negative user metrics including chronic lateness, internet addiction, social media addition, gaming addiction, and the like. For instance, the lifestyle analyzer component 104 may determine chronic lateness by determining an arrival time over threshold time after start time (e.g., arriving over 15 minutes after start time) over a number of time over a time period (e.g., late for more than 5 meetings over the last month). For instance, the lifestyle analyzer component 104 may determine social media addition based on time spent above a threshold time over a time period (e.g., more than 3 hours per day or 20 hours per week, etc.).

In various examples, the lifestyle analyzer component 104 may determine a stress level spike from sensor data and baseline metrics. In some examples, the lifestyle analyzer component 104 may determine the stress level fluctuation based on changes in heart rate from a heart monitor. In the present example, the stress level based on heart rate may be ignored if the user is exercising. In additional examples, the stress level may be further based on data received from additional sensor data (e.g., user's voice, user's words, user's facial expression, etc.). The stress level may be compared to a threshold (e.g., stress level>50) or may be based on a spike (e.g., stress level increases by more than 20 within a minute) to determine whether the user is stressed. In various examples, the system may provide a user interface to allow the user to manually self-report on health metrics, including but not limited to, general well-being, mood, stress level, energy level, and the like. The self-reported metrics may be any value or symbols that can be converted to value (e.g., on a scale of 1-5, smiling face to frowning face). The lifestyle analyzer component 104 may automatically trigger the user interface for self-reported metrics in response to a stress level spike, a negative health metric detection, a bedtime reminder, a morning reminder, an opening of specific application, and the like.

In various examples, the lifestyle analyzer component 104 can include functionality to present processed sensor data on a user interface on the user device(s) 102. For instance, the lifestyle analyzer component 104 may cause the user interface to present tracked data including current heart rate, resting heart rate, stress level, sleep time, current time, temperature, current location, steps taken, floors climbs, and active calories.

The lifestyle manager component 106 can include functionality to receive directives and to execute rules and filters to provide lifestyle management, as discussed herein. The lifestyle manager component 106 can include functionality to change a setting or apply a rule to change an application on the user device 102. In some examples, if the user and/or user profile accepted a directive (e.g., directive 116) from the concierge component 114, the lifestyle manager component 106 may implement the associated generated rule or download applicable application.

The user device(s) 102 can communicate with other user devices(s) 102 and/or one or more serving device(s) 110 via one or more network(s) 108.

In some instances, the serving device(s) 110 can include an aggregation component 112 and the concierge component 114.

The aggregation component 112 can receive user data and one or more metrics from the user device 102(1) and user device 102(2), as well as other user device, and aggregate the user data and metrics. The aggregation component 112 can aggregate the data based on user profile, time, data type, location, specific health metrics, and the like.

The concierge component 114 can analyze the aggregated data to determine an association and/or a correlation between a negative health metric (e.g., stress level spike, sleep debt, lack of movement, etc.) and user data to generate one or more recommendations and/or directives (e.g., directive 116) to provide personalize lifestyle improvement for the user. The concierge component 114, like a real-life concierge, may include a datastore of information, recommendations, and/or directives and may select the recommendations and/or directives to present to the user based on the analyzing the user data. In some instances, and as discussed herein, the directive 116 can be used, at least in part, to provide personalize lifestyle management for the user and/or user profile if accepted, to provide a rule to adjust an application setting, to perform a task based on meeting one or more criteria based on the rule, and the like. As can be understood, the concierge component 114 is not limited to outputting the directive 116, and any number of recommendations, directives, messages, or data can be utilized, as discussed herein.

Examples of the user device(s) 102 can include, but are not limited to, smart phones, mobile phones, cell phones, tablet computers, portable computers, laptop computers, personal digital assistants (PDAs), electronic book devices, or any other portable electronic devices that can generate, request, receive, transmit, or exchange voice, video, and/or digital data over a network. Additional examples of the user device(s) 102 include, but are not limited to, smart devices such as televisions, refrigerators, washing machines, dryers, smart mirrors, coffee machines, lights, lamps, temperature sensors, music players, headphones, or any other electronic appliances that can generate, request, receive, transmit, or exchange voice, video, and/or digital data over a network.

In some instances, the serving device(s) 110 can be implemented as one or more communication servers to facilitate communications by and between the various devices in the environment 100. That is, the serving device(s) 110 can represent any computing devices implementing various aspects of one or more of second, third, fourth generation, and fifth generation (2G, 3G, 4G, and 5G) cellular-wireless access technologies, which may be cross-compatible and may operate collectively to provide data communication services. Global Systems for Mobile (GSM) is an example of 2G telecommunications technologies; Universal Mobile Telecommunications System (UMTS) is an example of 3G telecommunications technologies; Long Term Evolution (LTE), including LTE Advanced, and Evolved High-Speed Packet Access (HSPA+) are examples of 4G telecommunications technologies; and New Radio (NR) is an example of 5G telecommunication technologies. Thus, the serving device(s) 110 may implement GSM, UMTS, LTE/LTE Advanced, and/or NR telecommunications technologies. In some instances, the telecommunication technologies can be referred to generally as a radio access technology. Thus, a 5G network can represent a 5G radio access technology.

The serving device(s) 110 may include, but is not limited to, a combination of: base transceiver stations BTSs (e.g., NodeBs, Enhanced-NodeBs, gNodeBs), Radio Network Controllers (RNCs), serving GPRS support nodes (SGSNs), gateway GPRS support nodes (GGSNs), proxies, a mobile switching center (MSC), a mobility management entity (MME), a serving gateway (SGW), a packet data network (PDN) gateway (PGW), an evolved packet data gateway (e-PDG), an Internet Protocol (IP) Multimedia Subsystem (IMS), or any other data traffic control entity configured to communicate and/or route data packets between the user device(s) 102, the serving device(s) 110, and/or the network(s) 108. In some embodiments, the serving device(s) 110 are operated by a service provider.

While FIG. 1 illustrates the serving device(s) 110, it is understood in the context of this document that the techniques discussed herein may also be implemented in other networking technologies, such as nodes that are part of a wide area network (WAN), metropolitan area network (MAN), local area network (LAN), neighborhood area network (NAN), personal area network (PAN), or the like.

Examples of the network(s) 108 can include, but are not limited to networks including second-generation (2G), third-generation (3G), fourth-generation (4G) cellular networks, such as LTE (Long Term Evolution), fifth-generation (5G) networks, and data networks, such as Wi-Fi networks. In some examples, the user device(s) 102 can transmit data on a preferred network(s) 108 based on network preferences. For instance, because the amount of user data gathered and transmitted to the serving device(s) 110 is continuous and may be high in volume, the user device(s) 102 may set a priority to use a faster Wi-Fi network if available and/or it may prioritize using the service provider's preferred network. In some examples, if multiple user devices 102 are in proximity to each other, and only a first user device 102 can connect to the service provider's preferred network, the other user device(s) 102 may attempt to tether to the preferred network through the first user device 102 or may transmit data to the first user device 102 and have data forwarded from there. In additional examples, a portion of the user data may be cached at each user device(s) 102 or at the first user device 102 until a low traffic time (e.g., during off-peak hour) for transmission.

In some instances, the user device(s) 102 can communicate with any number of user devices, servers, serving devices, computing devices, and the like.

In the present example, the example environment 100 can illustrate an example personalized lifestyle management based on user data collected from an example wearable (user device 102(1)) and an example user equipment (user device 102(2)). For example, the lifestyle analyzer component 104, on both user device 102(1) and user device 102(2), may gather and transmit user data via the network(s) 108 to the serving device(s) 110. The aggregation component 112 at the serving device(s) 110 may aggregate the user data. Based on the aggregated data, the serving device(s) 110 may identify a correlation between a stress level spike whenever the user and/or user profile associated with the user devices 102 receives a phone call from her Uncle John during her work hours. The concierge component 114 may generate a directive and a rule to push to the example user equipment. The lifestyle manager component 106 may present the directive to the user and/or user profile. If the user and/or user profile accepts the directive to screen calls from her uncle, the lifestyle manager component 106 may implement the rule to automatically screen calls from her Uncle John on Monday through Friday from 9 AM to 5 PM.

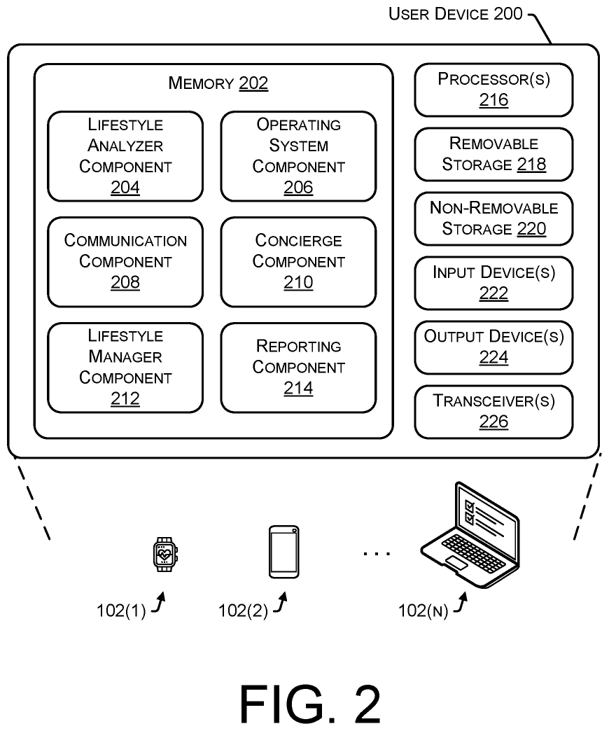

FIG. 2 illustrates an example user device 200 configured to implement the personalized lifestyle manager, in accordance with embodiments of the disclosure. In some embodiments, the user device 200 can correspond to the user device 102(1) and user device 102(2) of FIG. 1. It is to be understood in the context of this disclosure that the user device 200 can be implemented as a single device or as a plurality of devices with components and data distributed among them. By way of example, and without limitation, the user device 200 can be implemented as various user device 102(1), 102(2), . . . , 102(N).

As illustrated, the user device 200 comprises a memory 202 storing a lifestyle analyzer component 204, an operating system component 206, a communication component 208, a concierge component 210, a lifestyle manager component 212, and a reporting component 214. Also, the user device 200 includes processor(s) 216, a removable storage 218 and non-removable storage 220, input device(s) 222, output device(s) 224, and transceiver(s) 226.

In various embodiments, memory 202 is volatile (such as RAM), non-volatile (such as ROM, flash memory, etc.) or some combination of the two. The lifestyle analyzer component 204, the operating system component 206, the communication component 208, the concierge component 210, the lifestyle manager component 212, and the reporting component 214 stored in the memory 202 can comprise methods, threads, processes, applications or any other sort of executable instructions. The lifestyle analyzer component 204, the operating system component 206, the communication component 208, the concierge component 210, the lifestyle manager component 212, and the reporting component 214 can also include files and databases.

The lifestyle analyzer component 204 can include functionality to gather user data for analysis. In some examples, a user and/or user profile associated with the user device 200 may subscribe to a lifestyle management service or opt into providing user data for the service provider, and the service provider may provide the lifestyle analyzer component 204 to facilitate data gathering. The lifestyle analyzer component 204 may transmit user data to a remote serving device associated with the service provider for data analysis. In some examples, the lifestyle analyzer component 204 may perform data analysis locally at the user device 200 to provide at or near real-time feedback and directive for the user and/or user profile.

The lifestyle analyzer component 204 can include functionality to determine one or more health metrics associated with health data received, detected, or otherwise monitored by the user device 200, as described herein. For instance, the health data may be received from sensors including heart monitor, blood oxygen monitor, blood pressure monitor, scale, barometer, temperature sensor, pedometer, accelerometer, GPS, camera, microphone, and the like. Using the health data, the lifestyle analyzer component 204 can determine health metrics including but not limited to heart rate, hours slept, sleep quality, stress level, passive and active calories, steps taken, floors climbed, and the like for a user and/or user profile associated with the device.

In some examples, the user device 200 may include fitness tracking functionality and one or more sensors, and the lifestyle analyzer component 204 can instruct the operating system component to control software and/or hardware associated with the one or more sensors of the user device to monitor user's health data and to determine metrics indicative of the user's health conditions. Additionally, and/or alternative, the metrics may be indicative of the general conditions at or near the user (e.g., information about location, network environment, call status, traffic speed if driving, etc.). In some instances, information can be associated with the metrics, such as the geographical location of the user device (e.g., GPS or Wi-Fi based location), a cell identifier of a cell serving the user device, a signal-to-interference-plus-noise ratio (SINR), and the like. The lifestyle analyzer component 204 can send the metrics to a serving device to aggregate and analyze the metrics.

In some instances, the lifestyle analyzer component 204 can be implemented as an application operating on the user device 200. In some instances, to gather user data from other applications (e.g., social media, banking, calendar, etc.), the lifestyle analyzer component 204 can call an API (application programming interface) implemented in the operating system component 206, for example. Health data captured by sensors on a wearable device can be cross correlated to other user data from applications including calendar items, communications (e.g., phone calls, emails, etc.), location data, social media data, and financial data. Based on determined health metrics (e.g., heart rate, stress level, hours slept, steps taken, etc.), the lifestyle analyzer component 204 and the lifestyle manager component 212 may provide directives to automatically manage communications, schedules, and/or tasks.

In some instances, the lifestyle analyzer component 204 can include functionality to present a user interface on the user device 200, for example, to receive an indication from a user and/or user profile enabling or disabling a lifestyle analyzer component mode of the lifestyle analyzer component 204. For example, in some instances, a user and/or user profile of the user device can turn on or turn off the lifestyle analyzer component 204 based on personal preferences (or set privacy hours when data may not be tracked), incentives from a service provider, inclusion in a voluntary program, and the like.

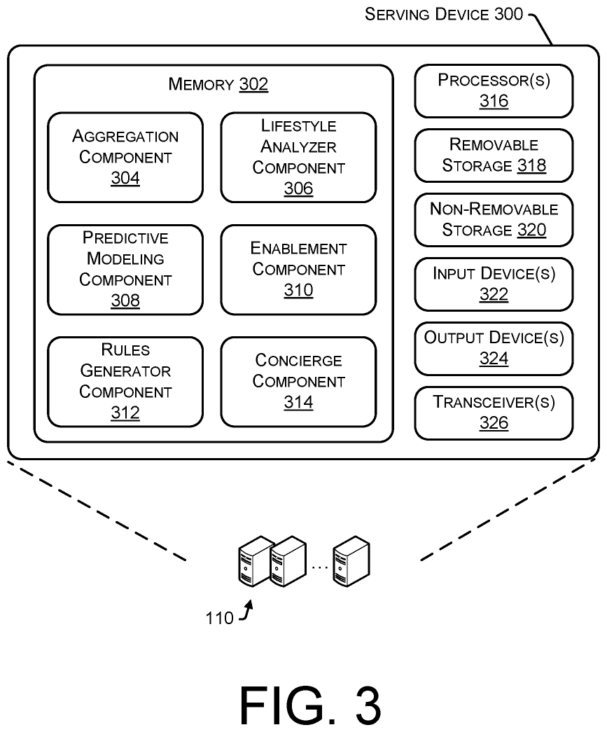

In some instances, the lifestyle analyzer component 204 can correspond to the lifestyle analyzer component 104 of FIG. 1 and/or lifestyle analyzer component 306 of FIG. 3.

The operating system component 206 can include functionality to instruct the software or hardware to gather and transmit data associated with a user and/or user profile of the user device 200, for example. In some instances, the operating system component 206 can include a first API to receive instructions from the lifestyle analyzer component 204 and the concierge component 210 and to provide data to the lifestyle manager component 212 and the reporting component 214. In some instances, the operating system component 206 can include a second API to issue instructions to software or hardware associated with a modem or the transceiver(s) 226 and to receive data from and/or transmit data to other user device 200 or serving device 300.

The communication component 208 can include functionality to conduct communications with one or more other devices, as discussed herein. In some instances, the communication component 208 can conduct communication with other user device 200 to provide a lifestyle management feedback loop at or near real-time. For instance, a first user device 200 with health tracking functions may track the stress level of the user, while a second user device 200 with map application running may remain in communication to determine to reroute the user based on stress level spike. In an additional example, a first user device 200 with health tracking functions may track the stress level of the user, while a second user device 200 with music application running may automatically change a music category based on the stress level of the user.

In some examples, the communication component 208 can conduct electronic communications (e.g., phone call, video chat, etc.) and store data related to the electronic communication for the lifestyle analyzer component 204. In various examples, the communication component 208 may interact with the lifestyle analyzer component 204 to determine when to transmit user data to the serving device for analysis. The communication component 208 may further determine whether a user device 200 is connected to a preferred network, which may be based on one or more prioritization schemes including using the fastest available network, using a device's preferred network (e.g., device hardware was designed to run faster on a 4G and/or 5G network), or using the network provided by the service provider. The communication component 208 may prioritize electronic communication over data transfer for lifestyle analyzer component 204. In some examples, the communication component 208 may allow multiple user devices 200 in vicinity of each other to tether or funnel data transfer through one of the user devices 200 based on the prioritization schemes.

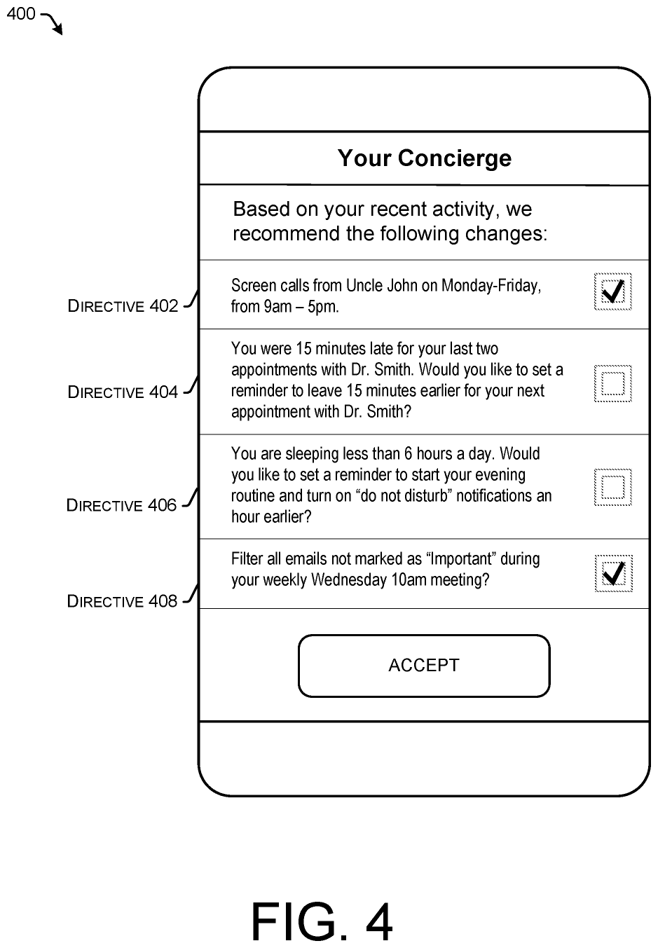

The concierge component 210 can include functionality to select and/or present lifestyle management directives on the user device 200. In some examples, the concierge component 210 may receive directives from the serving device or may generate directives locally and present the directives to the user. In additional examples, the concierge component 210 can provide a user interface to view and accept directives, as described herein with respect to FIG. 4. In examples, the concierge component 210 may prioritize the directives based on one or more predetermined user goals including increasing fitness, increasing productivity for school or work, increasing life balance, and increasing financial health. In some examples, the concierge component 210 may present a predetermined number of top ranked (e.g., top 5, top 10, etc.) directives for the user. The concierge component 210 may include rules to implement functionality for the directive include changing do not disturb notification, screening phone calls, holding texts, filtering emails, filtering electronic communications, restricting screen time, restricting application use, restricting website access, and the like. In various examples, the concierge component 210 may interact with lifestyle manager component 212 to implement the rules or provide recommended service based on whether the user accepted the directive. The concierge component 210 may track rules that were rejected or implemented by the user to send to the serving device to train models to predict which rules is more likely to be used by the user.

In some instances, the concierge component 210 can correspond to the concierge component 114 of FIG. 1 and/or the concierge component 314 of FIG. 3.

In some instances, the concierge component 210 can generate visualization for example directives as described herein with respect to FIG. 4.

The lifestyle manager component 212 can include functionality to change a setting or apply a rule to change an application on the user device 200. In some examples, if the user accepted a directive from the concierge component 210, the lifestyle manager component 212 may implement the associated generated rule or download applicable application. For example, the lifestyle manager component 212 can add reminders on the calendar application to perform accepted tasks (e.g., get ready for bed earlier, perform morning stretches, etc.). In additional examples, the lifestyle manager component 212 may present the generated rules for the user to further adjust. For instance, if the generated rule is an electronic communication filter or do not disturb setting for a certain time period (e.g., Monday-Friday, 9 am-5 pm, etc.) the time period may be presented for the user to change. In various examples, the lifestyle manager component 212 can download recommended applications (e.g., meditation application, workout application, etc.) and can present and/or automatically trigger the applications. In additional examples, the lifestyle manager component 212 can provide the recommended lessons including stretches, exercises, breathing exercise, and the like.

In some examples, the lifestyle manager component 212 may receive health metrics and change an application setting in response to the health metrics. For instance, if the user accepts a rule to automatically send early bedtime reminders based on sleep debt, the lifestyle manager component 212 may provide the early bedtime reminder based on determining the user slept less than 6 hours in the previous two nights. In another example, if the user accepts a rule to automatically set running tempo, the lifestyle manager component 212 may change a music tempo or songs based on determining the user needs to increase or decrease heart rate.

In some instances, the lifestyle manager component 212 can correspond to the lifestyle manager component 106 of FIG. 1.

The reporting component 214 can include functionality to store one or more metrics associated with the health or general status of a user of the user device 200 and to send such metrics to a serving device. In various examples, the reporting component 214 can send the one or more metrics to a serving device as the one or more metrics are generated, captured, or determined. In some instances, the reporting component 214 can aggregate the metrics and send the aggregated metrics to the serving device. In some instances, the reporting component 214 can send the metrics at a time of low network congestion (e.g., at night). In some instances, the reporting component 214 can encode the data sent to a serving device such that a service provider tracking data usage does not count the data against a quota associated with the user device 200.

In some embodiments, the processor(s) 216 is a central processing unit (CPU), a graphics processing unit (GPU), or both CPU and GPU, or other processing unit or component known in the art.

The user device 200 also includes additional data storage devices (removable and/or non-removable) such as, for example, magnetic disks, optical disks, or tape. Such additional storage is illustrated in FIG. 2 by removable storage 218 and non-removable storage 220. Tangible computer-readable media can include volatile and non-volatile, removable and non-removable media implemented in any method or technology for storage of information, such as computer readable instructions, data structures, program modules, or other data. Memory 202, removable storage 218 and non-removable storage 220 are all examples of computer-readable storage media. Computer-readable storage media include, but are not limited to, RAM, ROM, EEPROM, flash memory or other memory technology, CD-ROM, digital versatile discs (DVD), content-addressable memory (CAM), or other optical storage, magnetic cassettes, magnetic tape, magnetic disk storage or other magnetic storage devices, or any other medium which can be used to store the desired information and which can be accessed by the user device 200. Any such tangible computer-readable media can be part of the user device 200.

In various embodiments, the user device 200 can include applications including but are not limited, a web browser application, a video streaming application, an online gaming application, a lifestyle analyzer component, and the like. During execution on the user device(s) 102, each of the applications may be configured to cause the user device(s) 102 to initiate data communications with the serving device(s) 110 over the network(s) 108.

The user device(s) 102 may be configured to communicate over a telecommunications network using any common wireless and/or wired network access technology. Moreover, the user device(s) 102 may be configured to run any compatible device operating system (OS), including but not limited to, Microsoft Windows Mobile, Google Android, Apple iOS, Linux Mobile, as well as any other common mobile device OS.

The user device 200 also can include input device(s) 222, such as a keypad, a cursor control, a touch-sensitive display, voice input device, etc., and output device(s) 224 such as a display, speakers, printers, etc. These devices are well known in the art and need not be discussed at length here.

As illustrated in FIG. 2, the user device 200 also includes one or more wired or wireless transceiver(s) 226. For example, the transceiver(s) 226 can include a network interface card (NIC), a network adapter, a LAN adapter, or a physical, virtual, or logical address to connect to various network(s) 108, or to the serving device(s) 110, for example. To increase throughput when exchanging wireless data, the transceiver(s) 226 can utilize multiple-input/multiple-output (MIMO) technology. The transceiver(s) 226 can comprise any sort of wireless transceivers capable of engaging in wireless, radio frequency (RF) communication. The transceiver(s) 226 can also include other wireless modems, such as a modem for engaging in Wi-Fi, WiMax, Bluetooth, infrared communication, and the like.

FIG. 3 illustrates an example serving device 300 configured to receive user data captured by user device that includes lifestyle analyzer component, in accordance with embodiments of the disclosure. In some embodiments, the serving device 300 can correspond to the serving device(s) 110 of FIG. 1. It is to be understood in the context of this disclosure that the serving device 300 can be implemented as a single device or as a plurality of devices with components and data distributed among them.

As illustrated, the serving device 300 comprises a memory 302 storing an aggregation component 304, a lifestyle analyzer component 306, a predictive modeling component 308, an enablement component 310, a rules generator component 312, and a concierge component 314. Also, the serving device 300 includes processor(s) 316, a removable storage 318 and non-removable storage 320, input device(s) 322, output device(s) 324, and transceiver(s) 326.

In various embodiments, the memory 302 is volatile (such as RAM), non-volatile (such as ROM, flash memory, etc.) or some combination of the two. The aggregation component 304, the lifestyle analyzer component 306, the predictive modeling component 308, the enablement component 310, the rules generator component 312, and the concierge component 314 stored in the memory 302 can comprise methods, threads, processes, applications or any other sort of executable instructions. The aggregation component 304, the lifestyle analyzer component 306, the predictive modeling component 308 the enablement component 310, the rules generator component 312, and the concierge component 314 can also include files and databases.

The aggregation component 304 can include functionality to receive and aggregate user data from a user device, as discussed herein. The user data may include any data and/or metrics determined and/or captured by a user device. In some instances, the aggregation component 304 can receive a plurality of user data and store the user data in a database. In some instances, the user data can be indexed by location, data type, health metric type, time, user groups, goal groups, user device, user ID, user accounts, and the like. In various examples, the aggregation component 304 can aggregate the user data by any index (e.g., by health metric type, goal groups, or user ID, etc.).

In some instances, the aggregation component 304 can perform any statistical analysis on the metrics to determine a variety of corresponding data. The aggregation component 304 can aggregate user data from one or more devices to include health data and application data. For example, the aggregation component 304 can aggregate a user's health metrics determine the user exhibits elevated stress levels at approximately the same time every week. Using the time data, the aggregation component 304 can determine based on calendar data that the user is in a weekly team meeting during this time. The aggregation component 304 can also determine the user exhibited stress level spikes in response to incoming communications from family members during this time. The aggregation component 304 can determine family members from social media data or assigned phone book groups.

In another instance, the aggregation component 304 can identify a user group with fitness goals and the determine that a portion of the users are improving their fitness based on use of fitness applications. In the present example, the aggregation component 304 can further identify trends (e.g., favorite fitness application, favorite local gym, favorite meditation application, etc.) with the user groups and may determine directives based on the trends.

In some instances, the aggregation component 304 can correspond to the aggregation component 112 of FIG. 1.

The lifestyle analyzer component 306 can analyze aggregated data and determine directives, as discussed herein. The lifestyle analyzer component 306 can receive data processed by the aggregation component 304 and analyze any identified user trends, behavior patterns, or correlated user data to determine directives to provide for lifestyle management. In some examples, the lifestyle analyzer component 306 can identify negative health metrics (e.g., stress level spike) for a user and correlate user data to determine a cause for the negative health metrics. If a cause is identified, the lifestyle analyzer component 306 may determine if a directive can be provided to remedy the cause. In various examples, the lifestyle analyzer component 306 can determine data to collect from user devices for lifestyle analysis.

The predictive modeling component 308 can include functionality to predict areas in which the lifestyle management could help improve by providing directives. For example, the lifestyle analyzer component 306 can identify a large number of directives that could improve the user's lifestyle, however, not all the directive would align with the user's lifestyle goals. The predictive modeling component 308 can train one or more machine learning models to determine the likelihood of the directives being a desired improvement for the user and may track the acceptance rate and or success rate for each directive type to retrain the machine learning models. In some examples, the predictive modeling component 308 may group directives by directive types such as email filters, sleep reminders, or call screening to help track directives that may be too specific to track. In some examples, the predictive modeling component 308 may train with aggregated metrics to determine if an implemented directive provides a lifestyle improvement to determine a success rate and may adjust a weight associated with the directive to increase the likelihood of providing the same directive to another user. For instance, if a directive to use a particular fitness application demonstrates an increase in fitness level based on heart rate or weight metrics, the predictive modeling component 308 may increase the weight for recommending the particular fitness application.

The enablement component 310 can include functionality to enable a lifestyle analyzer component for individual user device. For example, the enablement component 310 can send invitations to various user device to determine if users of the user device wish to activate the lifestyle analyzer component, as discussed herein. In some instances, the enablement component 310 can enable the lifestyle analyzer component for individual user device based at least in part on characteristics of the user component, such as whether the user device is configured to receive sensor data associated with health metrics and whether user would provide permission to gather personal data from applications.

The rules generator component 312 can interact with the aggregation component 304 and include functionality to analyze the aggregated metrics to determining the rules that may be applied to a device of the user to provide lifestyle management, as discussed herein. The rules generator component 312 can generate rules to implement functionality for lifestyle management including downloading applications, changing do not disturb notification, screening phone calls, holding texts, filtering emails, providing alternate commute routes, adding reminders, and the like. In some examples, the rules generator component 312 can include sets of pre-generated rules that may be implemented based on the applications installed and/or the lifestyle goal. In various examples, the rules generator component 312 can modify parameters for the sets of rules to personalize it for the user based on the user's data (e.g., setting sleep timer based on user's usual bedtime). Using the example as described herein for the aggregation component 304, the rules generator component 312 can modify the parameters to filter incoming communications from family members during the weekly team meeting time.

In various examples, the rules generator component 312 can continuously monitor the user data to determine if additional adjustment is needed for the rules including adding additional communication filter, adjusting do not disturb setting.

The concierge component 314 can include functionality to select lifestyle management directives and push the directives to a user device, as discussed herein. The concierge component 314 may include datastores of knowledge database, product catalogs, application database, lifestyle management recommendations, lifestyle management directives, and the like. The concierge component 314 may select the recommendations and/or directives for a user profile based on the analyzing the associated user data. The concierge component 314 may select lifestyle management directives from sets of general directives that target different user goals. The concierge component 314 may select specific directives from the general directives based on user data and the user's goals including increasing fitness, increasing productivity for school or work, increasing life balance, and increasing financial health, and the like. In some examples, the concierge component 314 may select a predetermined number of top ranked (e.g., top 5, top 10, etc.) directives for the user. In some examples, the concierge component 314 may select directives based on weights applied to each directive and the weights may be adjusted based on the user data and goals. For instance, if the user's goal is to increase fitness and save money, a directive to walk and take public transportation may be weighted higher based on the two goals. The concierge component 314 may push the directives to the concierge component 210 on the user device associated with the user and may include the rules generated by the rules generator component 312 to implement the directives. In some examples, the concierge component 314 or 210 may message the user to approve implementation of the directives.

In some embodiments, the processor(s) 316 is a central processing unit (CPU), a graphics processing unit (GPU), or both CPU and GPU, or other processing unit or component known in the art.

The serving device 300 also includes additional data storage devices (removable and/or non-removable) such as, for example, magnetic disks, optical disks, or tape. Such additional storage is illustrated in FIG. 3 by removable storage 318 and non-removable storage 320. Tangible computer-readable media can include volatile and nonvolatile, removable and non-removable media implemented in any method or technology for storage of information, such as computer readable instructions, data structures, program modules, or other data. Memory 302, removable storage 318 and non-removable storage 320 are all examples of computer-readable storage media. Computer-readable storage media include, but are not limited to, RAM, ROM, EEPROM, flash memory or other memory technology, CD-ROM, digital versatile discs (DVD) or other optical storage, magnetic cassettes, magnetic tape, magnetic disk storage or other magnetic storage devices, or any other medium which can be used to store the desired information, and which can be accessed by the serving device 300. Any such tangible computer-readable media can be part of the serving device 300.

The serving device 300 can include input device(s) 322, such as a keypad, a cursor control, a touch-sensitive display, etc. Also, the serving device 300 can include output device(s) 324, such as a display, speakers, etc. These devices are well known in the art and need not be discussed at length here.

As illustrated in FIG. 3, the serving device 300 can include one or more wired or wireless transceiver(s) 326. In some wireless embodiments, to increase throughput, the transceiver(s) 326 can utilize multiple-input/multiple-output (MIMO) technology. The transceiver(s) 326 can be any sort of wireless transceivers capable of engaging in wireless, radio frequency (RF) communication. The transceiver(s) 326 can also include other wireless modems, such as a modem for engaging in Wi-Fi, WiMax, Bluetooth, infrared communication, or the like.

FIG. 4 illustrates an example visualization of personalized lifestyle manager based on user data captured using a lifestyle analyzer component, as discussed herein. In some instances, the example visualization can be represented as example concierge UI 400.

In some instances, the example concierge UI 400 can present various directives, recommendations, and/or messages including directive 402, directive 404, directive 406, and directive 408 in an environment. Further, the example concierge UI 400 can represent user interface buttons or other navigation elements to view additional directives and accept user input to select and/or deselect individual directives, as discussed herein.

In a first example, the directive 402 may provide an example directive for screening calls. The directive 402 may be generated in response to a stress level spike detected corresponding to a phone call received during work hour. If the directive 402 is accepted by the user, a follow-up user interface may be presented to the user to view the rule for screening calls and the user may change some of the settings. For instance, instead of screening calls from a specific caller like “Uncle John,” the user may select a category of people (e.g., relatives, family members, acquaintances, etc.) to screen calls.

In a second example, the directive 404 may provide an example directive for calendar reminder for a user that is chronically late. In the present example, the lifestyle manager may detect that the user is consistently late to a specific appointment and present directive for a reminder for the user to leave earlier. If the directive 404 is accepted by the user, a follow-up user interface may be presented to the user to view the rule for the reminder and the user may adjust how many minutes ahead of the appointment to be reminded.

In a third example, the directive 406 may provide an example directive for user with negative health metrics indicating sleep deprivation. In the present example, the lifestyle manager may detect that the user has not been getting enough sleep for a few days and may present directive to help the user get more sleep.

In a fourth example, the directive 408 may provide an example directive for filtering emails during an important weekly meeting. In the present example, the lifestyle manager may detect that the user has a longstanding weekly meeting and the user exhibits stress level spike whenever an email notification pops up. If the directive 408 is accepted by the user, a follow-up user interface may be presented to the user to view the rule for the email filter and the user may adjust the time window of the filter (e.g., 10 am-11 am, 10 am-12 pm, etc.).

In some instances, the example concierge UI 400 can be generated and presented by the concierge components 114, 210, and 314, as discussed herein. Of course, the example visualizations are not limited to the example concierge UI 400. Further, the directives 402, 404, 406, or 408 are not limited to communications filters and reminders and may include any directives and follow-up options for lifestyle management.

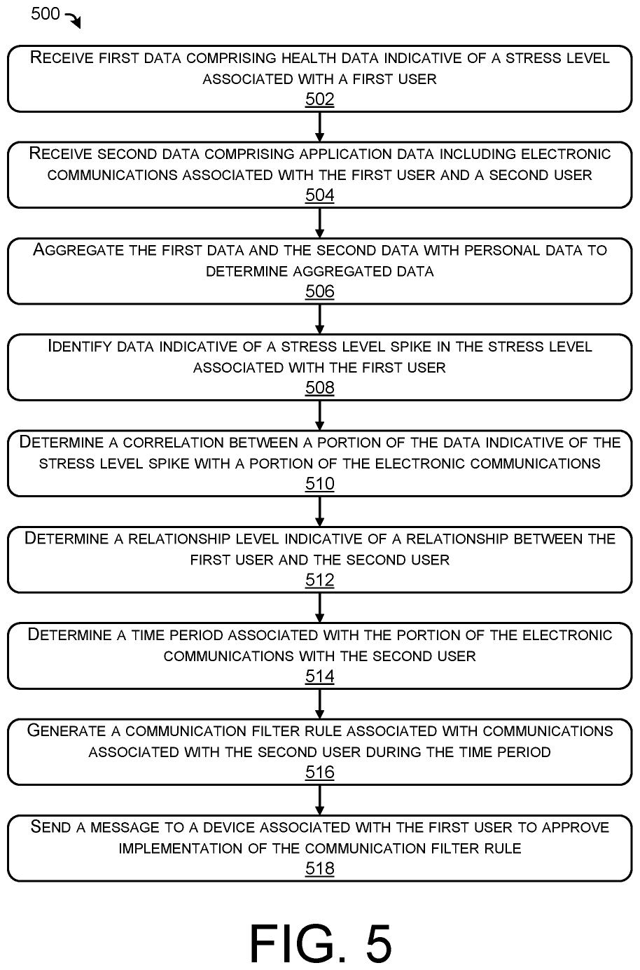

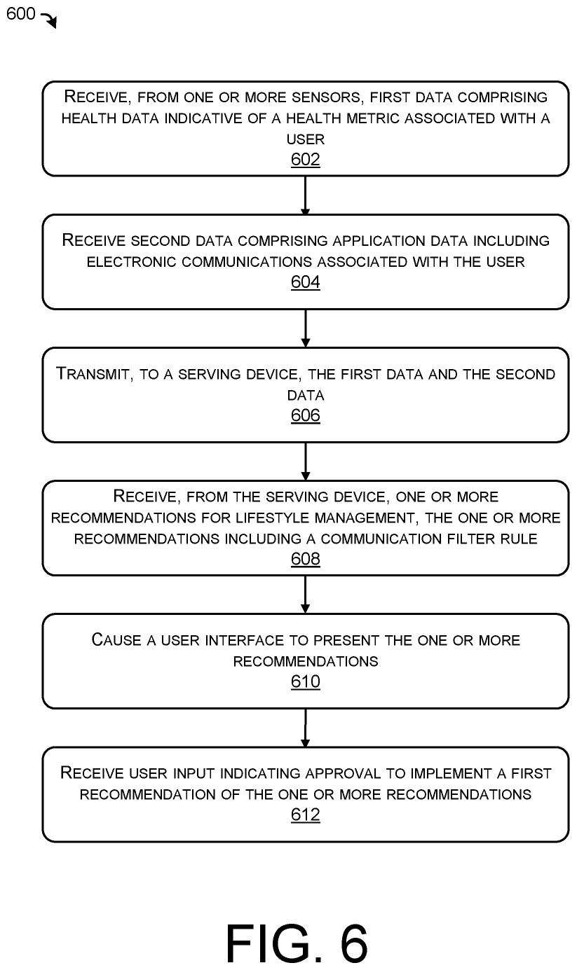

FIGS. 5 and 6 illustrate example processes in accordance with embodiments of the disclosure. These processes are illustrated as logical flow graphs, each operation of which represents a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the processes.

FIG. 5 illustrates an example process 500 for receiving user data from a wearable device and a user equipment, determining a stress level spike in response to an electronic communication, and generating a communication filter rule in response, as described herein. The example process 500 can be performed by the serving device(s) 110 and 300 (or another component), in connection with the user device(s) 102 and 200 (or another component), and other components discussed herein. Some or all of the process 500 can be performed by one or more devices, equipment, or components illustrated in FIGS. 1-3, for example.

At operation 502, the process can include receiving, from a wearable device, first data comprising health data indicative of a stress level associated with a first user. In some instances, the operation 502 can be performed by the user device using the lifestyle analyzer component 104 or 204, for example. In some instances, the lifestyle analyzer component can include functionality to receive and process sensor data from one or more sensors, as discussed herein. The sensor data may be received from one or more sensor that provide information on user's health metrics. In some instances, the lifestyle analyzer component can use one or more sensors to measure health data for various factors (e.g., general fitness, heart rate, stress level, sleep health, steps, calories burned, etc.) that affects user's health and lifestyle. The lifestyle analyzer component may determine a resting heart rate and/or a baseline stress level. In some examples, the lifestyle analyzer component may determine the stress level fluctuation based on changes in heart rate from a heart monitor. In the present example, the stress level based on heart rate may be ignored if the user is exercising. In additional examples, the stress level may be further based on data received from additional sensor data (e.g., user's voice, user's words, user's facial expression, etc.). The lifestyle analyzer component can send the metrics to a serving device to aggregate and analyze the metrics.

At operation 504, the process can include receiving, from a user equipment, second data comprising application data including electronic communications associated with the first user and a second user. In some instances, the operation 504 can receive, from a user equipment, user data from applications, sensors, or communications. The applications may include any category of applications including calendar, email, phone book, messenger, maps, social networking, games, media player, finance, fitness, lifestyle, shopping, and the like. The communications may be included with applications and may be functions native to phones including voice communications, video communications, texts, and the like. Although described as two devices (a wearable device and a user equipment), operations 502 and 504 may be received from a single user device such as a smart watch capable of tracking health metrics, placing calls, and storing various applications (e.g., email, calendar, phone book, banking applications, etc.).

At operation 506, the process can include aggregating the first data and the second data with personal data to determine aggregated data. In some instances, the operation 506 can be performed by a serving device using the aggregation component 112 or 304, for example. The aggregation component can include functionality to receive and aggregate user data from a user device, as discussed herein. The user data may include any data and/or metrics determined and/or captured by a user device. In some instances, the aggregation component can receive a plurality of user data and store the user data in a database. In some instances, the user data can be indexed by location, data type, health metric type, time, user groups, goal groups, user device, user ID, user accounts, and the like. In various examples, the aggregation component can aggregate the user data by any index (e.g., by health metric type, goal groups, or user ID, etc.).

At operation 508, the process can include identifying data indicative of a stress level spike in the stress level associated with the first user. In some instances, the operation 508 can be performed by a serving device using the aggregation component 112 or 304, for example. The aggregation component can aggregate user data from one or more devices to include health data and application data. In an example, the operation 508 can aggregate a user's health metrics determine the user exhibits elevated stress levels at approximately the same time every week. The operation 508 can also determine the user exhibited stress level spikes in response to incoming communications from family members during this time.

At operation 510, the process can include determining a correlation between a portion of the data indicative of the stress level spike with a portion of the electronic communications. In some instances, the operation 510 can be performed by a serving device using the aggregation component 112 or 304, for example. As described herein, and in the operation 508, the aggregation component can perform any statistical analysis on the metrics to determine a variety of corresponding data. The aggregation component can aggregate user data from one or more devices to include health data and application data. For example, the aggregation component can aggregate a user's health metrics determine the user exhibits elevated stress levels at approximately the same time every week. Using the time data, the aggregation component can determine based on calendar data that the user is in a weekly team meeting during this time. The aggregation component can also determine the user exhibited stress level spikes in response to incoming communications from family members during this time. The aggregation component can determine family members from social media data or assigned phone book groups.

At operation 512, the process can include determining, based at least in part on the aggregated data, a relationship level indicative of a relationship between the first user and the second user. The operation 512 can use the aggregated data to determine family members from social media data or assigned phone book groups.

At operation 514, the process can include determining a time period associated with the portion of the electronic communications with the second user. In some instances, the operation 514 can be performed by a serving device using the aggregation component 112 or 304, for example. As described herein, and in the operation 510, the aggregation component can perform any statistical analysis on the metrics to determine a variety of corresponding data. For example, the aggregation component can determine the user exhibits elevated stress levels at approximately the same time every week. Using the time data, the aggregation component can determine based on calendar data that the user is in a weekly team meeting during this time.

At operation 516, the process can include generating a communication filter rule associated with communications associated with the second user during the time period. In some instances, the operation 516 can be performed by a serving device using the rules generator component 312, for example. As described herein, rules generator component can analyze the aggregated metrics to determining the rules that may be applied to a device of the user to provide lifestyle management, as discussed herein. The rules generator component can include sets of pre-generated rules that may be implemented based on the applications installed and/or the lifestyle goal. In various examples, the operation 516 can modify parameters for the sets of rules to personalize it for the user based on the user's data (e.g., setting sleep timer based on user's usual bedtime). The operation 516 can modify the parameters to filter incoming communications from second user during the time period.

At operation 518, the process can include sending a message to a device associated with the first user to approve implementation of the communication filter rule. In some instances, the operation 518 can be performed by a serving device using the concierge component 314, for example. As described herein, concierge component can include functionality to select lifestyle management directives and push the directives to a user device, as discussed herein. The operation 518 may push the directives to the user device associated with the user and may include the rules generated to implement the directives. In some examples, the operation 518 may message the user to approve implementation of communication filter rule.

FIG. 6 illustrates an example process 600 for transmitting user data to a serving device and receiving directives to implement the personalized lifestyle manager, as described herein. The example process 600 can be performed by the user device(s) 102 and 200 (or another component), in connection with the serving device(s) 110 and 300 (or another component), and other components discussed herein. Some or all of the process 600 can be performed by one or more devices, equipment, or components illustrated in FIGS. 1-3, for example.

At operation 602, the process can include receiving, from one or more sensors, first data comprising health data indicative of a health metric associated with a user. In some instances, the operation 602 can be performed by the user device using the lifestyle analyzer component 104 or 204, for example. In some instances, the operation 602 can include functionality to receive and process sensor data from one or more sensors, as discussed herein. The sensor data may be received from one or more sensor that provide information on user's health metrics. In some instances, the operation 602 can use one or more sensors to measure health data for various factors (e.g., general fitness, heart rate, stress level, sleep health, steps, calories burned, etc.) that affects user's health and lifestyle.

At operation 604, the process can include receiving second data comprising application data including electronic communications associated with the user. In some instances, the operation 604 can receive, from the user device, user data from applications, sensors, or communications. The applications may include any category of applications including calendar, email, phone book, messenger, maps, social networking, games, media player, finance, fitness, lifestyle, shopping, and the like. The communications may be included with applications and may be functions native to phones including voice communications, video communications, texts, and the like.

At operation 606, the process can include transmitting, to a serving device, the first data and the second data. The operation 606 can include functionality to gather and transmit data to a serving device. For example, the operation 606 can store data related to the electronic communication and health metrics. In various examples, the operation 606 can determine when to transmit user data to the serving device for analysis. The operation 606 may further determine whether a user device is connected to a preferred network, which may be based on one or more prioritization schemes including using the fastest available network, using a device's preferred network (e.g., device hardware was designed to run faster on a 4G and/or 5G network), or using the network provided by the service provider. In some examples, the operation 606 may allow multiple user devices in vicinity of each other to tether or funnel data transfer through one of the user devices based on the prioritization schemes.

At operation 608, the process can include receiving, from the serving device, one or more directives for lifestyle management, the one or more directives including a communication filter rule. In some instances, the operation 608 can be performed by the concierge component 210 and/or 314, for example. In some examples, the operation 608 can include functionality to receive lifestyle management directives from a serving device. The operation 608 may include rules to implement functionality for the directive include changing do not disturb notification, screening phone calls, holding texts, and filtering emails.

At operation 610, the process can include causing a user interface to present the one or more directives. The operation 610 can include functionality to present lifestyle management directives on a user device. In additional examples, the operation 610 can provide a user interface to view and accept directives, as described herein with respect to FIG. 4.

At operation 612, the process can include receiving user input indicating approval to implement a first directive of the one or more directives. In some examples, the operation 612 can provide a user interface to accept directives, as described herein with respect to FIG. 4. As described herein, the process may include rules to implement functionality for the directive include changing do not disturb notification, screening phone calls, holding texts, and filtering emails. In various examples, the operation 612 can implement the rules or provide recommended service based on whether the user accepted the directive.

### CONCLUSION

Although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A system comprising:
one or more processors;
a memory; and
one or more components stored in the memory and executable by the one or more processors to perform operations comprising:
receiving, from a wearable device, first data comprising health data indicative of a stress level associated with a first user;
receiving, from a user equipment, second data comprising application data including electronic communications associated with the first user and a second user;
aggregating the first data and the second data with personal data to determine aggregated data;
identifying data indicative of a stress level spike in the stress level associated with the first user, wherein the stress level spike is an increase of the stress level above a threshold level within a predetermined time;
determining an association between a first portion of the data indicative of the stress level spike with a second portion of the electronic communications;
determining, based at least in part on the aggregated data, a relationship level indicative of a relationship between the first user and the second user;
determining a time period associated with the second portion of the electronic communications with the second user;
generating a communication filter rule associated with communications associated with the second user during the time period; and
sending a message to a device associated with the first user to approve implementation of the communication filter rule.

2. The system of claim 1, wherein the health data further includes at least one of:
heart rate data;
sleep data;
movement data; or
caloric consumption data.

3. The system of claim 1, wherein the personal data includes at least one of:
social media data;
location data;
image data;
streaming media data; and
financial data.

4. The system of claim 1, wherein the association is a first association, the stress level spike is a first stress level spike, and the time period is a first time period, the operations further comprising:
determining a second association between a second stress level spike with a second time period associated with a commute of the first user; and
determining a route associated with the commute; and
providing a second message associated with an alternate route.

5. The system of claim 4, the operations further comprising:
determining a third association between a stress level decrease and media presented during the second time period; and
providing a suggestion to present the media during the commute.

6. A method comprising:
receiving, from one or more user devices, personal data including health data and application data associated with a user profile;
identifying data indicative of a negative health metric in the health data;
determining an association between a first portion of the data indicative of the negative health metric with a second portion of the application data;
generating one or more directives to adjust an application setting based at least in part on the association; and
sending a message, to the one or more user devices, to approve implementation of the one or more directives.

7. The method of claim 6, wherein the one or more user devices include one or more of:
a wearable device;
a computing device; or
a cellphone.

8. The method of claim 6, wherein the health data includes at least one of:
stress level data;
heart rate data;
sleep data;
movement data; or
caloric consumption data.

9. The method of claim 6, further comprising:
associating one or more lifestyle goals with the user profile, wherein the one or more directives is further based at least in part on the one or more lifestyle goals.

10. The method of claim 9, wherein the one or more lifestyle goals includes one or more of:
fitness increase;
work productivity;
family harmony;
school productivity;
balanced mindset; or
financial fitness.

11. The method of claim 9, further comprising:
aggregating the personal data with other user data to determine aggregated user data;
identify additional users associated with the one or more lifestyle goals based at least in part on the aggregated user data; and
identify one or more approved directives for the additional users based at least in part on the aggregated user data, wherein the one or more directives is based at least in part on the one or more approved directives.

12. The method of claim 11, further comprising:
training a first machine learning model using the aggregated user data as first training data, wherein the one or more directives is generated using the first machine learning model;
receive additional personal data including a third portion of the one or more directives that was approved;
aggregating the additional personal data with the aggregated user data to determine second training data; and
training a second machine learning model using the second training data.

13. The method of claim 6, wherein the negative health metric is a stress level spike at a time period, the application data includes an electronic communication at the time period, and the one or more directives includes a communication filter rule during a time interval that includes the time period.

14. A method comprising:
receiving, from one or more sensors, first data comprising health data indicative of a health metric associated with a user profile;
receiving second data comprising application data including an electronic communication associated with the user profile;
transmitting, to a serving device, the first data and the second data;
receiving, from the serving device, one or more directives for lifestyle management, the one or more directives including a communication filter rule;
causing a user interface to present the one or more directives; and
receiving data indicative of an approval to implement a first directive of the one or more directives.

15. The method of claim 14, wherein the health metric indicates sleep deprivation and the one or more directives further includes an early bedtime reminder.

16. The method of claim 14, further comprising:
ranking the one or more directives based at least in part on a predetermined lifestyle goal; and
causing the user interface to present the one or more directives based at least in part on the ranking.

17. The method of claim 16, wherein the predetermined lifestyle goal indicates productive goals and the one or more directives further includes an electronic communication filter during work hours.

18. The method of claim 16, wherein the predetermined lifestyle goal indicates fitness goals and the one or more directives further includes a fitness application download.

19. The method of claim 14, wherein the application data including calendar data associated with the user profile and the health metric indicates stress level spike to chronic lateness and the one or more directives further includes an early appointment reminder.

20. The method of claim 14, wherein the health metric indicates high stress level and the one or more directives further includes a recommended breathing exercise.
